(19) United States
(12) Patent Application Publication
(10) Pub. No.: US 2008/0312491 A1
(43) Pub. Date: Dec. 18, 2008

(54) Sensory Apparatus

(76) Inventors: Kathryn Leigh Smith, Cornwall (GB); Jeremy John Smith, Cornwall (GB)

Correspondence Address: Kimberly A. Chasteen, PO Box 1243, Yorktown, VA, 23692, US

(21) Appl. No.: 11/916,698

(22) PCT Filed: Jun. 5, 2006
(86) PCT No.: PCT/GB06/02060

§ 371 Date: Aug. 13, 2008

(30) Foreign Application Priority Data
Jun. 6, 2005 (GB) 05114380.6

(51) Int. Cl. A61M 21/02

(52) U.S. Cl. 600/28; 600/27

(57) ABSTRACT

A highly mobile unit that will allow sensory room equipment to be stowed, moved and used in various locations.

The cart would allow provision of Sensory Integration and Sensory Processing therapies environments (often called ‘Snoezelen’) where costs and lack of space would prohibit development of a dedicated Sensory Room.

The cart will allow heavy, bulky and fragile equipment to be safely and easily moved en masse. It would be capable of being maneuvered into and around small spaces including hospital rooms and lifts. It will have an onboard rechargeable power supply, so that all audio-visual and sensory equipment will be able to be powered safely from within the unit, reducing risks of injury from leads, and ensuring equipment could always be used, even where no or inadequate power supply is available.

The configuration of Sensory Equipment on the cart will be capable of being customised to meet the needs of different client groups, and to meet national as well as local and specific Health and Safety, COSHH and Risk Reduction policies. The unit will be capable of being secured, so that all equipment can be safely stowed while moving the cart about or while it is not in use and is being stored.

The present invention relates to the management of arousal levels of patients, in particular to an apparatus and assembly for providing sensory treatment and aid to a patient to manage fluctuating levels of alertness, distress and agitation.

Currently well equipped specially designed Sensory Rooms are common in many hospital and care settings in order to be able to offer clients alternative strategies to manage altered states of arousal, including distress and agitation, assisting them to learn and develop practical coping strategies, reducing the need for invasive medical and physical interventions including sedating injections and Control and Restraint or Seclusion procedures. This approach offers an alternative strategy to increasing self management of clients with mental health difficulties who are distressed or angry, or engaging in self harm behaviours. The sensory approach assessment accurately predicts how clients will respond at a physiological and neuropsychological level to specific interventions. This allows clients to choose activities they enjoy using, while allowing therapists to then predict which interventions, including alternative therapies, will be effective at either raising or decreasing arousal levels, enabling clients to experience rapid success, promoting continued engagement with this approach. ‘Just Right’ sensory environments can then be created when the client is in crisis, according to the clients' needs.

Allocating space to be dedicated for use as a Sensory Room is becoming more difficult as services try to be flexible and provide more varied therapy options. To address this growing need for flexibility, portable Sensory Kits have been developed, these are often heavy, big and bulky, requiring many trips across sites to move equipment between storage areas and different buildings. Traditional ‘Sensory Carts’—old hospital trolleys stacked with plastic tubs of Sensory equipment, the obligatory battery operated CD Player and portable light projector—are just not good enough and look ‘home grown’ and unprofessional. As Health and Safety gets more stringent, how, when and where these homemade carts can be safely transported and used, means often clients cannot have access to equipment, or sometimes even if provided, it just cannot be powered in a convenient and, more importantly, safe manner. This restricts and denies clients therapeutic opportunities to learn how to use sensory strategies when distressed by emotions or events instead using more destructive ways, which can include harm of self or others.

Accordingly, there is a need to enable clients with mental health difficulties to have access to the therapeutic advantages of much of the equipment from a typical Sensory Room without services having to meet the high initial and ongoing expenses involved in provision of dedicated Sensory Rooms. The resource will ensure this therapy approach can be provided flexibly in a variety of locations where because of needing dedicated space, this type of approach would previously not be possible within existing/current room provision eg 136 De-escalation rooms, A and E Departments and maternity units.

According to the present invention, there is provided an apparatus for providing sensory stimulation to a subject, the apparatus comprising:
a housing, the housing being freely moveable across a surface;
a plurality of sensory stimulation means disposed within the housing and adapted for providing sensory stimulation to the environment surrounding the housing, the sensory stimulation means providing stimulation for one or more of the senses of touch, sight, smell, sound, taste, proprioception or vestibular;
wherein the apparatus is self contained.

The apparatus of the present invention is self contained, that is it comprises all the means necessary to provide sensory stimulation to a subject to achieve the required objective of therapy, calming, or the like.

The apparatus of the present invention is secure, that is the apparatus must be of a design and construction that reduces the risk of harm to a subject or other person to a minimum. In this respect, components that may be used as a weapon or a projectile are secured within the housing in general, in particular when the apparatus is not in use. Similarly, the exterior of the apparatus is of a design that minimises the risks of subjects using the apparatus as a whole to harm themselves or others.

The apparatus of the present invention is portable, being freely moveable across a surface, for example from room to room within a given facility. In this way, the sensory stimulation may be provided to subjects at their location. In addition, this avoids the need for having a dedicated room or space for providing such sensory treatment, as is currently the practice and requirement with existing technology. To render the apparatus fully moveable, the housing is preferably provided with castors or wheels of conventional design. The apparatus is most preferably moveable through 360°, in order to allow it to be moved into and out of tight and confined spaces.

Where Health and Safety guidelines are very strict, especially inpatient mental health units, the apparatus will be constructed to meet current Health and Safety guidelines. In particular, the apparatus should be constructed to avoid any sharp edges or protrusions, ligature points or damaging projectiles that may be used by a severely disturbed patient to inflict harm on themselves or others.

Accordingly, the housing is preferably provided with portions, such as doors, flaps or lids, which may be selectively opened to reveal the appropriate sensory stimulation means, while also allowing the sensory stimulation means to be secured out of reach of subjects, when not in use. The housing may be provided with handles or grabs, in order to ease moving and maneuvering the apparatus. If provided, the handles or grabs are removable or, most preferably, inset into the housing, so as not to protrude.

The housing is preferably provided with means for securing it in a closed position, for example a suitable lock or retaining device, such that the sensory stimulation means may be secured in an inaccessible manner, to avoid harming a subject when not in use.

In a particularly preferred embodiment, the housing is provided with one or more portions that open to reveal the sensory stimulation means and provide access thereto. In this way, the housing may be closed, in order to secure the components of the apparatus and prevent them from being damaged or being used to cause damage to themselves or others by a distressed subject.

Sensory stimulation means included in the apparatus can be customised to meet the needs of particular client group. The sensory stimulation means may be electrical or non-electrical. Suitable means are known in the art.

The sensory stimulation means are selected from means for stimulating the senses of sight, sound, smell, touch, taste, proprioception and vestibular. Preferably, the apparatus comprises means for stimulating a plurality of the aforementioned senses. A plurality of different means may be provided for stimulating a single sense. In a preferred embodiment, the apparatus comprising means for stimulating all of the aforementioned senses. Such an apparatus provides the most versatile embodiment and has the greatest range of therapeutic applications. However, alternative embodiments may have means for stimulating only some of the senses. The apparatus may comprise a single means or a plurality of means for stimulating a given sense. A single means may act as a stimulant for more than one sense.

To stimulate the visual sense of the subject, the apparatus may include a visual display means, such as an array of lights, for example a light emitting diode (LED) array, a plasma screen or liquid crystal display of known construction. Such a screen is preferably retained within the housing and adapted to be exposed to the subject, either by extending from within the housing or by having a portion of the housing open to reveal the screen. Additional visual stimuli may be provided by one or more light assemblies. These assemblies may act to provide lighting effects to the housing. In such a case, the housing may comprise one or more transparent or opaque portions, through which a light display may be viewed. Alternatively, or in addition thereto, the apparatus may comprise one or more assemblies for projecting light into the environment around the apparatus. Additional or alternative visual display means may be provided, for example electrically operated candles and the like. In this way, the apparatus may be used to provide an appropriate environment around a subject for treatment, for example by providing a softly lit environment to calm a disturbed or agitated subject.

In a preferred embodiment, the housing comprises one or a plurality of portions that may be opened, the visual stimuli being provided when the one or more portions of the housing are in the open position, the visual stimuli not being visible when the one or more portions of the housing are in the closed position. In this respect, the one or more portions of the housing may be provided with a transparent or opaque lining on its inner surface, with a suitable light assembly being located between the housing and the lining. A suitable light assembly for such use is an array of LEDs. The LEDs may be arranged in any suitable or desired pattern behind the lining, for example they may provide a lighting trim around the edge of the portion.

The visual stimuli may be adapted to be manipulated by a subject or a healthcare specialist. For example, the display screens may be of the touch-screen design, whereby subjects may interact with the display by touching the screen. Alternatively, the visual display may be adapted to be manipulated by a subject or other person by way of one of the other sensory stimulation means, for example the means for stimulating touch.

The visual stimuli may be provided in various formats. Such formats include calming displays, in which a visual display is adapted to provide a calming effect on a subject. In addition, the visual stimuli may be adapted to have one or more alert formats, in which the subject or other person is alerted and caused to respond. Any or all of these formats of visual display may be linked to cooperate with other sensory stimulation means, for example the means for generating aural stimuli.

Aural stimuli may be provided by a suitable means for projecting sounds from the apparatus. Suitable means for creating and projecting a sound are known in the art and include conventional apparatus for playing music or sounds recorded on conventional media, such as tape, compact disc, and computer readable memory. Other means that may be used to provide an aural display include sound emitting devices, such as shakers and the like. Such devices may require operation by the subject or another person.

The apparatus may also comprise means for stimulating the olfactory system of the subject. Means for producing scents and smells to treat and calm a subject are known in the art. Suitable means include known aromatherapy burners and the like.

In addition, the apparatus may comprise one or more means to stimulate the subject by touch. Again, suitable means for stimulating the touch senses of the subject are known in the art. Such means may be disposed within the housing, to be revealed when desired, or may be disposed on the outer surface of the housing. As noted hereinbefore, the means to stimulate the subject by touch may be arranged to activate or control other sensory stimulation means, for example the means for stimulating sight, aural or olfactory responses. Suitable means include a touch-activated screen and one or more pressure pads located on or within the housing. In this way, the subject may be caused or required to interact with the apparatus and the sensory stimuli.

In one embodiment, the apparatus is provided with one or more control means for activation and control of the sensory stimulation means. This may be any suitable processor or computer, with associated memory, such as RAM, ROM and/or hard disk data storage. Suitable processors for use in the apparatus are known in the art. The processor may be provided with all the sensory stimulation routines pre-programmed. Alternatively, or in addition, the processor may be provided with means to receive updated or modified routines, for example by way of a portable data storage device, such as a disk or chip, or by connection to a network, such as a local area network (LAN) or the internet.

As noted above, a key aspect of the apparatus of the present invention is that it is self-contained. In this respect, the apparatus requires no external supply of power or other resources in order to provide the full range of sensory stimuli required. In a preferred embodiment, one or more of the sensory stimulation means is powered electrically. In such a case, the apparatus must comprise an independent power supply. Such a power supply, for example a battery, must be disposed within the housing in such a way that access by unauthorized persons or subjects is restricted. For example, the independent power supply may be housed within a dedicated compartment within the housing.

The power storage and supply means is preferably rechargeable. The apparatus is preferably provided with suitable connections, for example an electrical socket, to allow recharging. Again, however, it is most preferred that such connections are not exposed on or project from the outer surface of the housing, in order to prevent harm coming to a subject or other person.

An onboard power supply means the apparatus can be safely used anywhere, with no risks of loose cables or the necessity of multiple vacant electrical sockets in places appropriate to locate and use equipment safely. The cart can be quickly moved and when necessary it can be removed from a room or be relocated to more appropriate settings, all equipment securely stowed inside, out of reach. In the case of an apparatus for use with subjects of lower risk of harm, the power storage means may be dispensed with and a conventional cable connection to a local power supply grid relied upon.

The housing may be constructed of any suitable material, including metal, plastic and/or wood. One preferred material is wood, for example birch or other form of plywood. An alternative preferred material is plastic, in particular moulded plastic. Plastic or wood housings have the advantage of providing improved stimulation to touch, compared with metal housings. The housing may be covered in part or wholly with a coating or covering material. For example, the housing may have a soft washable vinyl or other padded covering. The covering or outer surface of the housing may comprise part or all of the means for stimulating the sense of touch. The housing may be painted or varnished.

Embodiments of the present invention will now be described, by way of example only, with reference to the accompanying drawings in which:

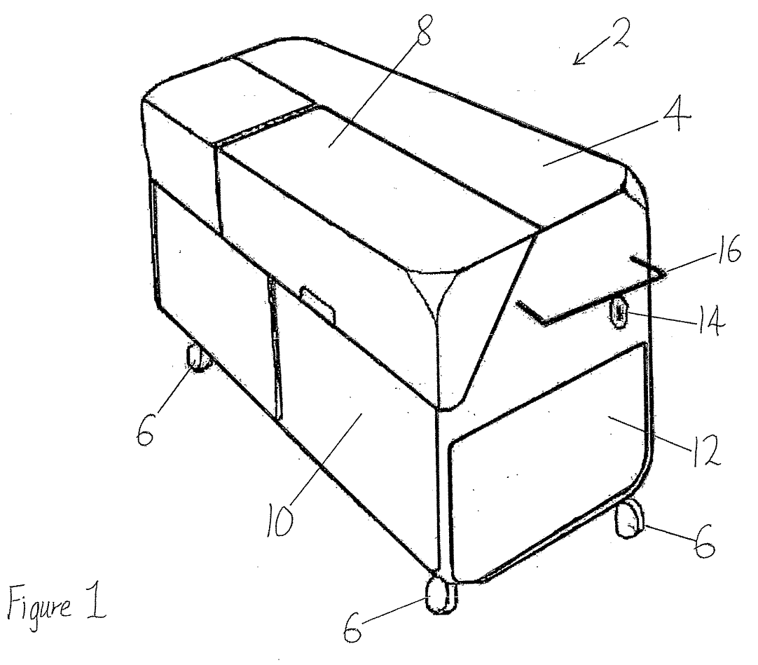

FIG. 1 is a perspective view of a first embodiment of the apparatus of the present invention in a closed position;

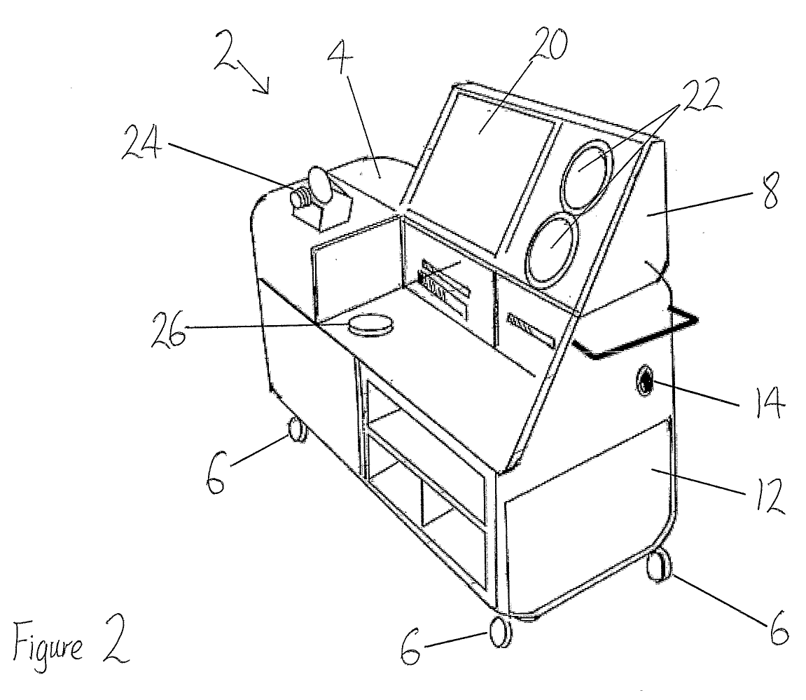

FIG. 2 is a perspective view of the apparatus of FIG. 1 in an open position;

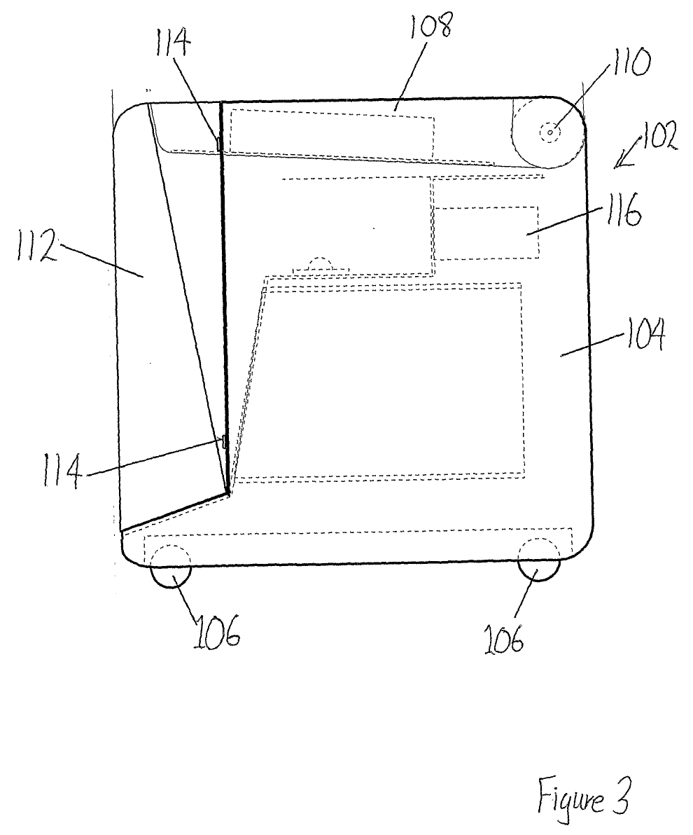

FIG. 3 is a side elevation of a second embodiment of the apparatus of the present invention in a closed position;

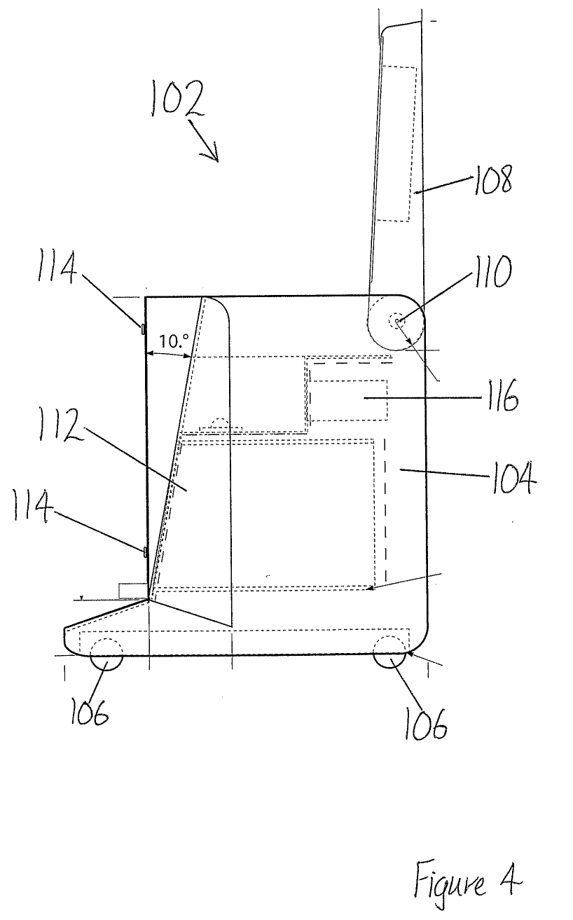

FIG. 4 is a side elevation of the apparatus of FIG. 3 in an open position;

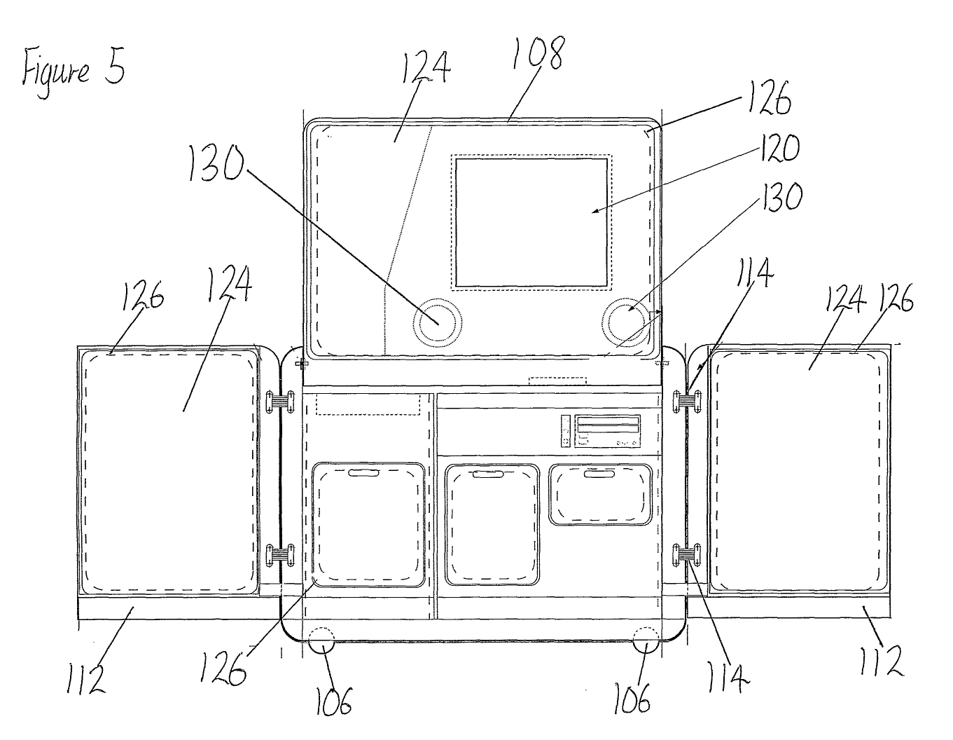

FIG. 5 is a front elevation of the apparatus shown in FIG. 4 along the line V-V; and

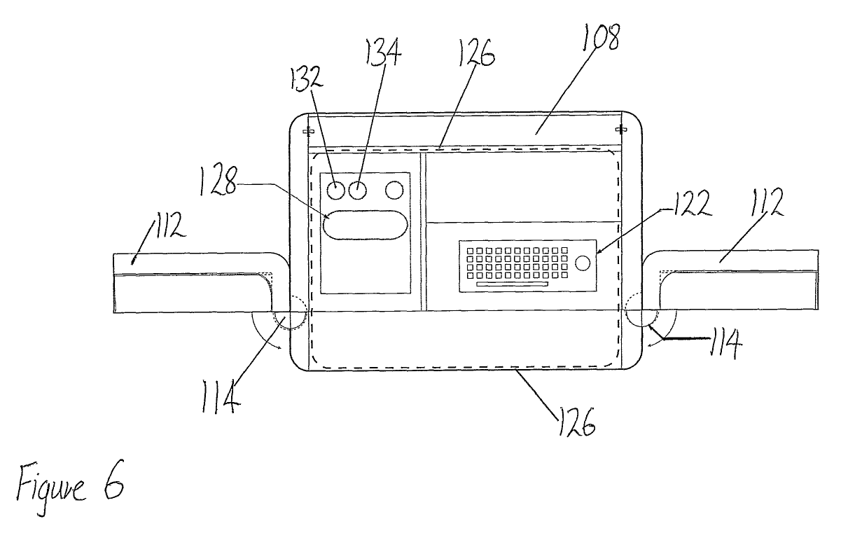

FIG. 6 is a plan view of the apparatus shown in Figure along the line VI-VI.

Referring to FIG. 1, an apparatus according to a first embodiment of the present invention is shown and generally indicated as 2. The apparatus comprises a generally rectangular housing 4 supported by castors 6 of conventional design. The housing 4 is provided with rounded edges and corners, and a smooth outer surface, in order to prevent injury or harm to a disturbed subject or other person.

The housing 4 has a hinged front portion 8, which may be raised to open the housing to access the interior, as shown in FIG. 2. The housing 4 further comprises a sliding front portion 10, which may be opened to gain further access to the interior, as shown in FIG. 2.

A compartment 12 is provided in one end of the housing 4 to house a battery (not shown for clarity). A socket 14 is provided in the end of the housing above the compartment 12 to provide a connection for charging the battery by means of a conventional cable and plug assembly, which may be stored in the housing when not in use. The socket 14 is flush with the outer surface of the housing 4.

As shown in FIG. 1, the apparatus 2 is provided with a handle 16 at one end. The handle shown in FIG. 1 extends from the housing. The handle 16 is preferably retractable within the housing. An alternative and preferred embodiment provides a handle or grab assembly that is recessed into the housing, again to avoid harming a subject or other person. In a further alternative, the handle or grab may be omitted, as shown in FIG. 2.

As shown in FIG. 1, when closed the overall approximate preferred dimensions of the apparatus are typically 1200 mm long, 1000 mm high and 700 mm deep. This will allow it to be transported in lifts and moved through doorway and along disabled ramps. The apparatus may be sized according to the duty to be performed and the needs of the particular facility or environment in which it will be used.

Referring to FIG. 2, the apparatus 2 of FIG. 1 is shown in an open, operating mode. Thus, the front portion 8 is open, revealing a visual display screen 20 and a pair of speakers 22. As in FIG. 2 a visual display unit (VDU) 20 and speakers 22 will be housed separately in the apparatus to ensure the VDU and speakers are protected in storage or while the apparatus is being pushed around.

Electrical equipment (which can be customized according to a particular unit or client groups needs) can be plugged into the battery and will then be mounted to the upper surfaces of the housing, either permanently or just while in use. Thus, a light projector 24 is shown in FIG. 2 mounted to the upper surface of the housing 4 at one end, which may be operated to provide specific lighting effects in the room or environment of the apparatus. Similarly, an aromatherapy assembly 26 for providing olfactory stimulation in the room or environment is shown mounted within the housing and exposed for use when the front portion 8 is in the raised or open position.

Referring to FIGS. 3 to 6, there are shown views of an apparatus according to a second embodiment of the present invention. Referring to FIG. 3, the apparatus, generally indicated as 102, comprises a housing 104 supported on a set of castors 106. The housing 104 has all edges and corners rounded to avoid injury or harm to subjects or other persons. The housing 104 has an interior divided into a plurality of compartments and sections, illustrated by broken lines in FIGS. 3 and 4. A battery (not shown for clarity) is housed within one of the lower compartments and is provided with a connection and socket (not shown) to allow for charging from a remote power source, such as a conventional domestic or industrial electrical supply.

The housing 104 comprises a lid portion 108, moveable about hinges 110, so as to open the entire top portion of the housing. A pair of front portions 112 are pivoted about hinges 114 and may be opened to expose the entire front of the interior of the housing 104. The housing in its open position is shown in FIGS. 4 to 6.

The housing 104 is provided with a cut out handle 116 (no ligature risk) on either side to allow the apparatus to be pushed or pulled by 1 or 2 people. Alternatively, the cut out handle may be provided in the ends of the lid portion 108.

Referring now to FIGS. 5 and 6, the sensory stimulation assemblies of the apparatus and their arrangement will now be described. Referring to FIG. 5, a visual stimulation means comprise several separate components. First, an LCD screen 120 is mounted to the inner surface of the lid portion 108. The screen 120 may be used to display still or moving images, including videos. The operation of the screen 120 is controlled by a keyboard 122 of conventional design mounted in the upper interior of the housing, as shown in FIG. 6. The apparatus may comprise suitable data storage means for the images to be displayed in the screen 120, such as computer readable memory, compact disk (CD), digital video disk (DVD), tape or the like.

Second, light assemblies are disposed on the interior surfaces of the lid portion 108 and front portions 112 of the housing, together with interior surfaces that are exposed and visible when the housing is opened. The light assemblies a mounted behind an opaque or transparent lining 124 secured to the inside surface of the lid portion 108 and the front portions 112. As shown in FIGS. 5 and 6, the light assemblies comprise strips of LEDs 126. Other suitable forms of light assemblies may also be employed.

Further, fibre optic cables may be provided to project light patterns from within the apparatus, for example located in a projection assembly 128 mounted in the upper interior of the housing 104.

Aural stimulation is provided by a pair of speakers 130, mounted to the inner surface of the lid portion 108 of the housing. The speakers 130 may be controlled using the keyboard 122. Again, suitable data storage means are provided to store sound data, as hereinbefore described, and including MP3 storage and playback means.

Other sensory stimulation means may be provided, as required by the duty to be performed. In particular, olfactory stimulation means 132, for example aromatherapy vapouriser assemblies, may be provided. These are mounted in the upper interior portion of the housing. Similarly, means for stimulating the vestibular, taste and/or proprioception senses may be provided.

Touch sense stimulation means 134 may also be provided, again mounted in the upper interior portion of the housing. In the embodiment shown in FIGS. 5 and 6, the region containing the fibre optic display 128, the olfactory stimulation means 132 and the touch sense stimulation means 134 may be used as an interactive therapy tool for use with subjects.

1. An apparatus for providing sensory stimulation to a subject, the apparatus comprising:
a housing, the housing being freely moveable across a surface;
a plurality of sensory stimulation means disposed within the housing and adapted for providing sensory stimulation to the environment surrounding the housing, the sensory stimulation means providing stimulation for one or more of the senses of touch, sight, smell, sound, taste, proprioception or vestibular; where the apparatus is self contained.

2. An apparatus according to claim 1, wherein the housing is provided with movement devices selected from the group consisting of wheels and castors.

3. An apparatus according to claim 1, wherein the housing is provided with a grasping device selected from the group consisting of handles and grabs.

4. An apparatus according to claim 3, wherein the grasping device is recessed into the housing, so as not to protrude from the plane of the surface of the housing, or are removeable.

5. An apparatus according to claim 1, wherein the housing is provided with a portion that may be opened or removed to access the interior of the housing.

6. An apparatus according to claim 5, wherein the said portion forms at least part of the top of the housing.

7. An apparatus according to claim 1, wherein the sensory stimulation means comprises a means for stimulating the visual senses of a subject.

8. An apparatus according to claim 7, wherein the said means comprises a visual display screen.

9. An apparatus according to claim 8, wherein the visual display screen is selected from the group consisting of a plasma screen and a liquid crystal display.

10. An apparatus according to claim 7, wherein the means for stimulating the visual senses of a subject comprises a light assembly.

11. An apparatus according to claim 10, wherein the light assembly is operable to light a portion of the housing.

12. An apparatus according to claim 10, wherein the light assembly is operable to project into the surrounding environment.

13. An apparatus according to claim 1, wherein the sensory stimulation means comprises a means for stimulating the aural sense of a subject.

14. An apparatus according to claim 13, wherein the said means comprises at least one speaker for projecting a sound.

15. An apparatus according to claim 14, wherein the at least one speaker is arranged to project sound through a portion of the housing or is exposed by opening a portion of the housing.

16. An apparatus according to claim 1, wherein the sensory stimulation means comprises means for stimulating the olfactory sense of a subject.

17. An apparatus according to claim 16, wherein the means for stimulating the olfactory sense of a subject is adapted to provide aromatherapy.

18. An apparatus according to claim 1, wherein the sensory stimulation means comprises means to stimulate the sense of touch, vestibular or proprioception of a subject.

19. An apparatus according to claim 1, comprising means for stimulating a plurality of senses, the means for stimulating a first sense being linked with the activation of the means for stimulating a second sense.

20. An apparatus according to claim 1, wherein one or more of the means for stimulating the sense requires interaction with a subject or other person.

21. An apparatus according to claim 1, comprising a means for storing and/or providing power to one or more of the sensory stimulation means.

22. An apparatus according to claim 21, wherein the means for storing and providing power is a battery.

23. An apparatus according to claim 21, wherein the means for storing and providing power is rechargeable.
